(19) United States
(12) Patent Application Publication
(10) Pub. No.: US 2021/0325372 A1
(43) Pub. Date: Oct. 21, 2021

(54) METHOD TO ASSESS CAR FUNCTIONALITY

(71) Applicant: INVECTYS SA, Paris (FR)

(72) Inventors: Maria LOUSTAU, Paris (FR); François ANNA, Bourg La Reine (FR); Pierre LANGLADE DEMOYEN, Neuilly-sur-Seine (FR); Julien CAUMARTIN, Le Vesinet (FR)

(21) Appl. No.: 17/271,767

(22) PCT Filed: Aug. 30, 2019
(86) PCT No.: PCT/EP2019/073262

§ 371 Date: Feb. 26, 2021

(30) Foreign Application Priority Data
Aug. 31, 2018 (EP) 18306152.2

(51) Int. Cl. G01N 33/50; G01N 33/58; G01N 33/92

(52) U.S. Cl. CPC G01N 33/5094; G01N 33/92; G01N 33/582

(57) ABSTRACT

The present invention relates a method for assessing the functionality of a chimeric antigen receptors (CAR) expressing cell. More specifically, this method is based on a trogocytosis process which involves membrane transfer between antigen expressing target cells and antigen specific immune cells.

### FIELD OF THE INVENTION

The invention relates to the field of immunology, cell biology, and molecular biology. In certain aspects, the field of the invention concerns immunotherapy. More particularly, the invention relates to a method for determining chimeric antigen receptor (CAR) characteristics such as specificity, functionality, and sensitivity.

### BACKGROUND OF THE INVENTION

For years, the foundations of cancer treatment were surgery, chemotherapy, and radiation therapy. However, in the past several years, immunotherapy has emerged as an effective tool in cancer treatment.

Advances in genetic engineering have led to the design of synthetic tumor targeting receptors, termed chimeric antigen receptors (CARs) that can be introduced into human immune cells such as T cells, to redirect antigen specificity and enhance functions of effector immune cells. CARs were first developed in the mid-1980s and the interest on these receptors is growing since. They were first generated to bypass the intrinsic TCR specificity of expressing T cells by providing specific antigen recognition independently from HLA-peptides complexes. Prototypic single chain CARs were first described in a study by Eshhar and colleagues in 1993, in which specific activation and targeting of T cells was mediated through molecules consisting of a target-antigen-specific antibody domain and the γ- or ζ-signaling subunits of the Fc epsilon receptor or T-cell receptor CD3 complex, respectively (Eshhar et al., 1993, Proc Natl Acad Sci., 90, 720-4). Since then, many groups have devised CAR molecules with single tumor-directed specificities and enhanced signaling endodomains. Nowadays, the binding domain of a CAR typically consists of an antigen-binding domain of a single-chain antibody (scFv) or antibody-binding fragment (Fab) selected from a library and comprising the light and heavy chain variable fragments of a monoclonal antibody (Mab) joined by a flexible linker. The scFv retains the same specificity and a similar affinity as the full antibody from which it was derived and is able to specifically bind to the target antigen of interest. CARs thus combine antigen-specificity and T cell activating properties in a single fusion molecule. Indeed, the scFv is linked to an intracellular signaling module that includes CD3ζ to induce T cell activation upon antigen binding. The modular structure has been extended from first-generation CARs with only a CD3ζ signaling domain to second and third generation CARs that link the signaling endodomains such as CD28, 4-1BB, or OX40 to CD3ζ in an attempt to mimic co-stimulation.

CARs have allowed T cells to be targeted against cancers in an MHC independent mechanism. Ligand binding of a CAR differs from that of a TCR binding to peptide/MHC (pMHC) in receptor affinity, antigen density, and spatial properties; and experimental approaches to design an optimal CAR for a specific target molecule have relied on functional assays of transduced T cells in vitro or in human tumor xenograft models. Because it is unlikely that CARs will serially engage target molecules and cluster in organized synapses as it is observed with TCR/pMHC recognition, it is assumed that a higher ligand density is required for CAR recognition than for TCRs. The optimal configuration and application of these receptors, besides affinity and specificity, rely on construct design, signaling domains, vector delivery systems, recipient immune-cell populations, and manufacturing.

Particularly, CARs effectiveness depends in part on the affinity of the CAR itself and, on the other part, on the components of the ectodomain. The presence of flexible linker sequences in the scFv and the type of connection between the ecto- and the endodomain (hinge and transmembrane regions) can alter CAR function profoundly by modifying (i) the length and the flexibility of the resulting CAR, (ii) its cell surface density, (iii) its tendency to self-aggregate and produce T cell exhaustion by tonic signaling, and (iv) its potential binding to molecules other than the intended target antigen.

Thus, CAR specificity and function can be affected by its structure (CAR length and epitope distance, spacer, transmembrane and endodomain regions) and its sensitivity (antibody affinity, antigen density/avidity, signal thresholds). However, there are few limited studies and assays designed to address this question and selections of optimal antibodies or CAR constructs are still not evidenced, which generally leads to the assessment of CAR function in a partial way, while these characteristics are of great interest to improve CAR-based therapies.

Methods of testing a CAR for the ability to recognize target cells and for antigen specificity are known in the art, for example by measuring the release of cytokines (Clay et al, J. Immunol., 163: 507-513 (1999)) or by measuring cellular cytotoxicity (Zhao et al, J. Immunol., 174: 4415-4423 (2005)). However, these methods can be time consuming and difficult to implement. There is thus a need to develop a simple, fast and effective method to assess CAR expressing cells functionality.

### SUMMARY OF THE INVENTION

To overcome this problem, the inventors developed a method to assess CAR expressing cells functionality. This method is based on trogocytosis, which involves membrane transfer between antigen expressing target cells and CAR expressing cells. This assay allows to evaluate CAR-effector cell activation in an original rapid way, depending on both the structure and the sensitivity of the recombinant CAR structure.

The present invention concerns an in vitro method for assessing the functionality of chimeric antigen receptor (CAR) expressing cells, comprising:
Labelling target antigen expressing cells and CAR expressing cells with different labels, wherein the target antigen expressing cells and the CAR expressing cells are prepared from the same cell line; Co-incubating the labelled target cells and the labelled CAR expressing cells,
Analyzing the cells in order to assess membrane acquisition by the CAR expressing cells from the target antigen expressing cells, the membrane acquisition being indicative of the binding and/or activation capacity of the CAR expressing cells to the target antigen, thereby assessing the functionality of the CAR expressing cells,
preferably wherein the CAR expressing cells are immune cells.

Preferably, the co-incubation is performed at least 1 hour, preferably during 1 to 5 hours, even more preferably during 1 to 3 hours. Particularly, the co-incubation is performed in a 1:0.5 to a 1:10 target antigen expressing cells to CAR expressing cells ratio.

The cells analysis is performed by cell sorting analysis, preferably flow-cytometry analysis.

Preferably, the in vitro method for assessing the functionality of chimeric antigen receptor (CAR) expressing cells further comprises a step of incubating target antigen expressing cells with an antibody that recognizes the same or an overlapping epitope compared to the antibody from which the CAR is derived before co-incubating the labelled target antigen cells and the labelled CAR expressing cells. Such antibody is preferably a monoclonal antibody, preferably a monoclonal antibody that comprises CDRs of the monoclonal antibody from which the CAR is derived.

Preferably, the in vitro method for assessing the functionality of chimeric antigen receptor (CAR) expressing cells further comprises testing control cells that do not express the targeted antigen. Such method may further comprise testing control CAR expressing cells that do not recognize the targeted antigen. Such method may also further comprising the selection of the functional CAR expressing cells and/or CAR construct.

The labels are membrane markers, preferably lipophilic tracers or dyes, even more preferably selected from the group consisting of MINI26, PKH26-PCL, PKH67, MINI67, PKH67-PCL, PKH26, PKH26-PCL, Vybrant CM-Dil, Dil and DiO.

Preferably, the cells are immune cells, preferably selected from T lymphocytes, B lymphocytes, natural killer cells, natural killer T cells, monocytes and antigen presenting cells.

The invention also relates to an in vitro method for selecting functional CAR expressing cells for adoptive cell therapy, which method comprises:

(i) transducing the population of immune cells with a nucleic acid sequence encoding a CAR, preferably wherein the population of immune cells has been isolated from a biological sample from a subject to be treated,

(ii) selecting a subpopulation of said isolated cells expressing the CAR and,

(iii) assessing their functionality by the method according to any of the preceding claims, preferably towards target antigen expressing cells that expresses the antigen for which the CAR has been generated

(iv) optionally selecting the functional CAR expressing cells and/or the CAR nucleic acid construct from the CAR expressing cells that experienced the membrane acquisition form the antigen expressing cells.

Preferably, the antigen is HLA-G, even more preferably HLA-G1.

The invention also concerns a cell, preferably an immune cell, comprising the functional CAR construct selected by any in vitro method disclosed herein.

### BRIEF DESCRIPTION OF THE DRAWINGS

FIG. 1: Membrane of target cells, Jurkat and HLA-G1/b2M expressing cell lines were labeled in green using the PKH67 dye. Membrane of effector cells, Jurkat HLA-G-15E7 and HLA-LFTT-1 CAR cells, were labeled in red using the PKH26 dye. Cells were then co-incubated at a 1:1 ratio and collected after 0, 1 and 3 hours of co-incubation before being analyzed by flow-cytometry.

FIG. 2: Membrane acquisition from tumor cells (labeled in green) by HLA-G CAR cells (labeled in red): (A) no membrane acquisition is expected by HLA-G CAR cells after co-incubation with HLA-G negative tumor cells, whereas (B) after co-incubation with HLA-G1/b2M expressing tumor cells, only anti-HLA-G1/b2M CAR cells should acquire membrane patches.

FIG. 3: Membrane acquisition from either Jurkat or HLA-G1/b2M Jurkat tumor cells by HLA-G CAR cells was investigated by flow-cytometry. HLA-G-15E7 CAR cells (left panel) did not exhibit PKH67 either after 1 and 3 hours of co-incubation with Jurkat or HLA-G1/b2M Jurkat cells. HLA-G-LFTT-1 CAR cells (right panel) displayed PKH67 at their surface only after co-incubation with HLA-G1/b2M Jurkat cells which extent increased overtime. Representative of 3 independent experiments.

FIG. 4: Kinetic of membrane acquisition from either Jurkat or HLA-G1/b2M Jurkat tumor cells by HLA-G-15E7 or HLA-G-LFTT-1 CAR cells (n=3).

FIG. 5: Membrane acquisition from HLA-G1/b2M Jurkat tumor cells by HLA-G-LFTT-1 CAR cells is prevented by pre-incubating tumor cells with LFTT-1 monoclonal antibody.

FIG. 6: (A) HLA-G-LFTT-1 CAR specificity was evaluated in the presence of the anti-HLA-G LFTT-1 monoclonal antibody (representative of 3 independent experiments). (B) HLA-G-LFTT-1 CAR specificity is related to the monoclonal antibody LFTT-1 paratope used for its generation (n=3).

FIG. 7: (A) Jurkat, Jurkat HLA-G1 and HLA-G-LFTT-1 CAR cells conjugates were evaluated in presence of the anti-HLA-G LFTT-1 monoclonal antibody (representative of 4 independent experiments). (B) Jurkat HLA-G1 and HLA-G-LFTT-1 CAR conjugates are prevented by the LFTT-1 monoclonal antibody (n=4).

FIG. 8: Schematic representation of membrane transfer assay between HLA-G negative (Jurkat cells in red), HLA-G positive (Jurkat HLA-G1 cells in green) target cells and effector cells (HLA-G-LFTT-1 CAR cells in blue).

FIG. 9: (A) Representative figure of membrane acquisition between HLA-G tumor expressing Jurkat cells and HLA-G CAR effector cells (B) Membranes acquired by HLA-G CAR effectors are only transferred from HLA-G expressing tumor cells and not from surroundings HLA-G negative tumor cells demonstrating that HLA-G CR activation is only driven by HLA-G expressing tumor cells and not by surroundings HLA-G negative tumor cells,

FIG. 10: Membrane acquisition from HLA-G1 target cells by effector CAR cells is not dependent on the membrane dye used.

FIG. 11: Schematic representation of the differential membrane transfer assay between HLA-G negative, HLA-G positive target cells and effector CAR cells.

### DETAILED DESCRIPTION OF THE INVENTION

### Introduction

The inventors developed a new, original, easy and rapid method for determining CAR characteristics such as specificity, function and sensitivity. This method is based on trogocytosis process which involves membrane transfer between antigen expressing target cells and antigen specific immune cells. This method allows to determine these characteristics in less than one day by using steps that can easily carried out in a laboratory. An important aspect for the effectiveness and simplicity of the method is to use the same cell line for preparing the CAR expressing cells and the target antigen expressing cells.

Accordingly, the present invention relates to an in vitro method for assessing the functionality of chimeric antigen receptors (CAR) expressing cells, comprising labelling target antigen expressing cells and CAR expressing cells with different labels, the target antigen expressing cells and the CAR expressing cells belonging to the same cell line; co-incubating the labelled target cells and the labelled CAR expressing cells and analyzing the cells in order to assess membrane acquisition by the CAR expressing cells from the target antigen expressing cells, the membrane acquisition being indicative of the binding and/or activation capacity of the CAR expressing cells to the target antigen.

Abbreviations

| | |
|---|---|
| APC: Antigen Presenting Cell | ICP: Immune CheckPoint |
| β2M: β-2-Microglobulin | ITAM: Immunoreceptor |
| | Tyrosine-based |
| | Activation Motif |
| CAR: Chimeric Antigen | NK: Natural Killer |
| Receptor | |
| CTL: Cytotoxic T | MHC: Major |
| Lymphocyte | Histocompatibility |
| | Complex |
| DC: Dendritic Cell | scFv: single-chain |
| | variable Fragment |
| HLA-G: Human Leukocyte | SD: Standard Deviation |
| Antigen G | |

### Definitions

To facilitate the understanding of the invention, a number of terms are defined below.

The terms “Chimeric antigen receptor” (CAR), “engineered cell receptor”, “chimeric cell receptor”, or “chimeric immune receptor” (ICR) as used herein refer to engineered receptors, which graft an antigen binding specificity onto immune cells (e.g. T cells or NK cells), thus combining the antigen binding properties of the antigen binding domain with the immunogenic activity of the immune cell, such as the lytic capacity and self-renewal of T cells. Particularly, a CAR refers to a fused protein comprising an extracellular domain able to bind an antigen, a transmembrane domain, optionally a hinge domain and at least one intracellular domain. The terms “extracellular domain able to bind an antigen”, “external domain”, “ectodomain” and “antigen binding domain” are used interchangeably herein and mean any oligopeptide or polypeptide that can bind to a targeted antigen. Particularly, the term “antigen binding domain” or “antigen-specific targeting domain” as used herein refers to the region of the CAR which targets and binds to specific antigens. When a CAR is expressed in a host cell, this domain forms the extracellular domain (ectodomain) of the receptor. The antigen binding domain of a CAR typically derives from an antibody and may consist of an antigen-binding domain of a single-chain antibody (scFv) or antigen-binding fragments (Fab). The terms “intracellular domain”, “internal domain”, “cytoplasmic domain” and “intracellular signaling domain” are used interchangeably herein and mean any oligopeptide or polypeptide known to function as a domain that transmits a signal that causes activation or inhibition of a biological process in a cell. The intracellular signaling domain may generate a signal that promotes an immune effector function of the cell transduced with a nucleic acid sequence comprising a CAR, e.g. cytolytic activity and helper activity, including the secretion of cytokines. The term “transmembrane domain” means any oligopeptide or polypeptide known to span the cell membrane and that can function to link the extracellular and signaling domains. This may be a single alpha helix, a transmembrane beta barrel, a beta-helix of gramicidin A, or any other structure. Typically, the transmembrane domain denotes a single transmembrane alpha helix of a transmembrane protein, also known as an integral protein. A chimeric antigen receptor may optionally comprise a “hinge domain” which serves as a linker between the extracellular and transmembrane domains. As used herein the terms “hinge”, “spacer”, or “linker” refers to an amino acid sequence of variable length typically encoded between two or more domains of a polypeptide construct to confer for example flexibility, improved spatial organization and/or proximity. The term “linker” as used in the context of a scFv refers to a peptide linker that consists of amino acids such as glycine and/or serine residues used alone or in combination, to link variable heavy and variable light chain regions together. A chimeric antigen receptor may optionally comprise a signal peptide. The terms “signal peptide” “targeting signal”, “localization signal”, “transit peptide” or “leader sequence” refer to a short peptide present at the N-terminus of the majority of newly synthesized proteins that are destined towards the secretory pathway. The core of the signal peptide may contain a long stretch of hydrophobic amino acids. The signal peptide may or may not be cleaved from the mature polypeptide.

As used herein, the terms “antibody” and “antibodies” refer to monoclonal antibodies, polyclonal antibodies, multispecific antibodies, human antibodies, humanized antibodies, camel antibodies, chimeric antibodies, single-chain variable fragment (scFv), single chain antibodies, single domain antibodies, antigen-binding fragments (Fab), F(ab′) fragments, disulfide-linked variable fragment (sdFv), intrabodies, nanobodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgGi, IgG₂, IgG₃, IgG₄, IgAi and IgA₂) or subclass. Unless specifically noted otherwise, the term “antibody” includes intact immunoglobulins and “antibody fragments” or “antigen binding fragments” that specifically bind to an antigen of interest to the substantial exclusion of binding to other molecules. The term “antibody” also includes genetically engineered forms such as chimeric antibodies (for example, humanized murine antibodies), heteroconjugate antibodies (such as, bispecific antibodies). Preferably, the term antibody refer to a monoclonal antibody, even more preferably to a scFv derived from a monoclonal antibody.

In terms of structure, an antibody may have heavy (H) chains and light (L) chains interconnected by disulfide bonds. There are two types of light chain, lambda (λ) and kappa (κ). Each heavy and light chain contains a constant region and a variable region (or “domain”). Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The framework regions act to form a scaffold that provides for positioning the CDRs in correct orientation by inter-chain, non-covalent interactions. The CDRs are primarily responsible for binding to an epitope of an antigen. The CDRs of each chain are typically referred to as CDR1, CDR2, and CDR3, numbered sequentially starting from the N-terminus. The VL and VH domain of the antibody according to the invention may comprise four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively. These framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. These framework regions and complementary determining regions are preferably operably linked in the following order: FR1-CDR1-FR2-CDR2-FR3-CDR3-FR4 (from amino terminus to carboxy terminus).

An “antibody heavy chain” as used herein, refers to the larger of the two types of polypeptide chains present in antibody conformations.

An “antibody light chain,” as used herein, refers to the smaller of the two types of polypeptide chains present in antibody conformations, K and X light chains refer to the two major antibody light chain isotypes.

The term “scFv” refers to a protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein a scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences.

The term “derive from” or “derived from” as used herein refers to a compound or protein having a structure derived from the structure of a parent compound or protein and whose structure is sufficiently similar to those disclosed herein and based upon that similarity, would be expected by one skilled in the art to exhibit the same or similar properties, activities and utilities as the claimed compounds. For example, a scFv derived from a monoclonal antibody refers to an antibody fragment that share the same properties that the monoclonal antibody, e.g. share identical or similar VH and VL or CDRs and/or recognize the same epitope.

In the context of cell, the term “derive from” or “derived from” as used herein refers to one or several cells that are prepared from a cell of the same cell line, for instance by genetic engineering such as infection or transformation. In particular, a target antigen expressing cell can be prepared by introducing into a cell of the cell line a nucleic acid encoding the antigen to be expressed.

Similarly, a CAR expressing cell can be prepared by introducing into a cell of the cell line a nucleic acid encoding the CAR.

As used herein, the term “competitive antibody” refers to an antibody that recognize the same or an overlapping epitope that is recognized by another antibody. When related to CAR, a competitive antibody may be the antibody or antibody fragment from which the CAR is derived or any antibody that recognizes the same or the overlapping epitope which the CAR recognizes.

As used herein, the term “antigen” or “target antigen” refers to a compound, composition, or substance that may be specifically bound by the products of specific humoral or cellular immunity, such as an antibody molecule, a T-cell receptor or a CAR. It is readily apparent that the present invention includes intact antigen and antigen fragment thereof. An antigen can be generated synthesized or can be derived from a biological sample. Such a biological sample can include, but is not limited to a tissue sample, a tumor sample, a cell or a biological fluid.

As used herein, the term “HLA-G” and “Human leukocyte antigen G” refers to a specific molecule associated with this name, including but not limited to any one of its several isoforms, including by not limited to membrane-bound isoforms (e.g., HLA-G1, HLA-G2, HLA-G3, HLA-G4), soluble isoforms (e.g., HLA-G5, HLA-G6, HLA-G7), and soluble forms generated by proteolytic cleavage of membrane-bound isoforms (e.g. sHLA-G1).

As used herein, “bind” or “binding” refer to peptides, polypeptides, proteins, fusion proteins and antibodies (including antibody fragments) that recognize and contact an antigen. Preferably, it refers to an antigen-antibody type interaction. By “specifically bind” or “immunospecifically bind” it is meant that the antibody recognize a specific antigen, but does not substantially recognize or bind other molecules in a sample. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope). As used herein, the term “specific binding” means the contact between an antibody and an antigen with a binding affinity of at least 10⁻⁶ M.

The affinity of an antibody can be a measure of its bonding with a specific antigen at a single antigen-antibody site, and is in essence the summation of all the attractive and repulsive forces present in the interaction between the antigen-binding site of an antibody and a particular epitope. The affinity of an antibody to a particular antigen may be expressed by the equilibrium constant K, defined by the equation K=[Ag Ab]/[Ag][Ab], which is the affinity of the antibody-combining site where [Ag] is the concentration of free antigen, [Ab] is the concentration of free antibody and [Ag Ab] is the concentration of the antigen-antibody complex. Where the antigen and antibody react strongly together there will be very little free antigen or free antibody, and hence the equilibrium constant or affinity of the antibody will be high. Particularly, a high affinity is a binding affinity of at least 10⁻⁶ M. The binding affinity can be measured by any method available to the person skilled in the art, in particular by surface plasmon resonance.

A “stimulatory ligand,” as used herein, means a ligand that when present on an antigen presenting cell (e.g., an APC, a dendritic cell, a B-cell, and the like) can specifically binds with a cognate binding partner (referred to herein as a “stimulatory molecule”) on a T cell, thereby mediating a primary response by the T cell, including, but not limited to, activation, initiation of an immune response, proliferation, and the like.

The term “co-stimulatory ligand” as used herein, includes a molecule on an antigen presenting cell (e.g., an APC, dendritic cell, B cell, and the like) that specifically binds with a cognate binding partner (referred to herein as a “stimulatory molecule”) on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like.

As used herein, a “co-stimulatory molecule” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as an immunoreceptor tyrosine-based activation motif or ITAM. Particularly this term refers to the cognate binding partner on a T cell that specifically binds with a co-stimulatory ligand present on an antigen presenting cell, thereby mediating a co-stimulatory response by the T cell, such as, but not limited to, proliferation activation, differentiation, and the like.

A “stimulatory molecule,” as used herein, means a molecule on a T cell that specifically binds with a cognate stimulatory ligand present on an antigen presenting cell.

A “co-stimulatory signal”, as used herein, refers to a signal, which in combination with a primary signal, such as TCR/CD3 ligation, leads to T cell proliferation activation, differentiation, and the like, and/or upregulation or downregulation of key molecules.

By the term “stimulation” or “stimulatory” is meant a primary response induced by binding of a stimulatory molecule (e.g., a TCR/CD3 complex) with its cognate ligand thereby mediating a signal transduction event, such as, but not limited to, signal transduction via the TCR/CD3 complex, Stimulation can mediate altered expression of certain molecules, such as downregulation of TGF-β, and/or reorganization of cytoskeletal structures, and the like.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). In particular, the immune cell can be selected in the non-exhaustive list comprising B cell, T cell, in particular CD4⁺ T cell and CD8⁺ T cell, NK cell, NKT cell, APC cell, dendritic cell, and monocyte.

The term “CAR expressing cell” or “CAR-cell” refers to a cell, particularly an immune cell (e.g. a T cell) that is genetically engineered to produce CARs on its surface. CAR-cells are generally generated to target a specific antigen that is expressed on cancer cells. Upon tumor cells recognition by CAR-antigen binding, the CAR-cells are activated to kill the cancer cells.

The terms “target antigen expressing cell” or “antigen expressing cells”, as used herein, refer to a cell, particularly an immune cell (e.g. T cell) that presents an antigen of interest on its surface, particularly an antigen identified as to be expressed on tumor cells for example. These cells can be genetically engineered to produce the antigen or alternatively naturally produce the antigen on their surface.

The term “control cells” refer to cells, preferably immune cells (e.g. T-cells), that can be used as a control, either because they don't express the CAR (hereafter “control CAR-cells”) or the antigen of interest (hereafter “control cells”). Preferably, they belong to or are prepared/derived from the same cell line than the cell line used for CAR expressing cells and/or the target antigen expressing cell.

The term “cell line” as used herein refer to a homogenous population of cells that shares the same genetic properties.

The term “label” or “marker” refers to any detectable label in a cell, particularly an immune cell, including radioactive label and non-radioactive label. Non-radioactive labels include optically detectable labels, such as for example fluorescent, luminescent or phosphorescent dyes labels or dyes. Labels include directly detectable and indirectly detectable labels. The term “fluorescent label”, as used herein, refers to any label detectable via fluorescent emission of the label, for example, via fluorescent spectroscopy. The label or marker is a dye that labels the membrane of the cells such as a lipid dye.

The term “cell sorting” refers to a method used to separate cells according to their type and/or characteristics. Cells are mostly commonly separated relying on differences in cell size, shape (morphology), and/or surface protein or marker expression. This method may result to the obtaining of homogeneous population of cells. Cells sorting can rely on different strategies known by the man in the art such as single cell sorting, fluorescent cell sorting, magnetic cell sorting or buoyancy activated cell sorting. Preferably, this term refer to Fluorescent Activated Cell Sorting, or FACS, that utilizes flow cytometry to provide a fast, objective and quantitative measurement of intra- and extracellular properties such as the expression of a fluorescent label, for sorting cells populations.

The term “functionality of the CAR expressing cells” as used herein, refer to the capacity of a cell, preferably of an immune cell (e.g. T-cell), to fulfill its biological function. When related to a CAR-cell, the functionality refers to the capacity of recognizing a target cell through binding an antigen (i.e., the antigen for which the CAR-cell has been generated), and to the activation capacity of the CAR expressing cell, (e.g. the capacity of activating the signaling pathway which leads to the killing of target cells expressing the antigen (e.g. via cytolytic activity and helper activity, including the secretion of cytokines)). Accordingly, the term “functional CAR-cells” as used herein, refers to CAR expressing cell able to bind a targeted antigen upon which the activation of the signaling pathway may be effectively induced.

The term “co-incubation” as used herein refer to the act or process of incubating at least two molecules or cells, preferably immune cells. In the context of the invention, the co-incubation of cells may lead to the contact of cells, particularly of two or more populations of cells such as the CAR-cell and the antigen expressing cells and optionally control cells.

The term “transfected” or “transformed” or “transduced” are used interchangeably herein and are applied to the production of chimeric antigen receptor cells and particularly refer to the process whereby a foreign or exogenous nucleotide sequence is introduced into a cell. The exogenous nucleic acid may be introduced stably or transiently into the host cell. A “transfected” or “transformed” or “transduced” cell is one which has been transfected, transformed or transduced with exogenous nucleic acid. In some embodiments, this transduction is performed via a vector, preferably a lentiviral vector.

The term “adoptive cell therapy” or “adoptive T-cell therapy” or “ACT” as used herein means the transfer of cells into a patient, where the cells have been engineered to or otherwise altered prior to transfer into the subject. An example of ACT is the harvesting from a subject's blood or tumor, an immune cell, such as a T cell. These immune cells are then stimulated ex vivo, in culture and expanded. The cells are then transduced with one or more nucleic acid constructs that allow the cell to express new molecules, such as a CAR, providing the engineered immune cells with a new mechanism for combating a disease, for instance a cancer. In some instances, the CAR comprise an antigen binding domain that specifically recognizes an antigen expressed by a tumor or cancer, such as HLA-G. Typical immune cells utilized in ACT procedures include tumor-infiltrating lymphocytes (TIL) or T cells. Immune cells used in ACT can be derived from the patient/subject themselves, or from a universal donor. ACT may also be accompanied by the optional step of lympho-depletion of the subject's own lymphocytes that may compete with the recombinant cells infused back into the subject.

As used herein, the term “subject”, “host”, “individual,” or “patient” refers to human and veterinary subjects particularly to an animal, preferably to a mammal, even more preferably to a human, including adult and child. However, the term “subject” also encompasses non-human animals, in particular mammals such as dogs, cats, horses, cows, pigs, sheep and non-human primates, among others.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such term refers to the improvement or eradication of a disease, a disorder, an infection or symptoms associated with it. In other embodiments, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect.

As used herein, the terms “nucleic acid construct”, “plasmid”, and “vector” are equivalent and refer to a nucleic acid molecule that serves to transfer a passenger nucleic acid sequence, such as DNA or RNA, into a host cell. A vector may comprise an origin of replication, a selectable marker, and optionally a suitable site for the insertion of a sequence or gene. A vector can be either a self-replicating extrachromosomal vector or a vector which integrates into a host genome. It can also comprise expression elements including, for example, a promoter, the correct translation initiation sequence such as a ribosomal binding site and a start codon, a termination codon, and a transcription termination sequence. A nucleic acid construct may also comprise other regulatory regions such as enhancers, silencers and boundary elements/insulators to direct the level of transcription of a given gene. Vectors capable of directing the expression of genes and/or nucleic acid sequence to which they are operatively linked can also be referred to herein as “expression vectors”. There are several common types of vectors including nucleic acid constructs, bacterial virus genomes, phagemids, virus genomes, cosmids, and artificial chromosomes. The nucleic acid construct can be a vector for stable or transient expression of a gene or sequence. The nucleic acid construct may comprise a nucleic acid construct origin of replication (ori). Particularly, the nucleic acid construct may be designed for genetic transfer between different hosts, including but not limited to a plasmid, a virus, a cosmid, a phage, a BAC, a YAC. Preferably, the nucleic acid construct encodes a CAR.

Method for Assessing the Functionality of CAR Expressing Cells

The invention concerns an in vitro method for assessing the functionality of chimeric antigen receptors (CAR) expressing cells, comprising (i) labelling of target antigen expressing cells and CAR expressing cells with different labels, wherein the target antigen expressing cells and the CAR expressing cells are prepared from the same cell line (ii) co-incubating the labelled target cells and the labelled CAR expressing cells (iii) analyzing the cells in order to assess membrane acquisition by the CAR expressing cells from the target antigen expressing cells, the membrane acquisition being indicative of the binding capacity of the CAR expressing cells to the target antigen, thereby assessing the functionality of the CAR expressing cells, and (iv) optionally, selecting the functional CAR expressing cells and/or the functional CAR construct. These steps are more particularly described hereafter.

Cells

The cell according to the invention is a eukaryotic cell, such as mammalian cells, and typically are human, feline or canine cells, more typically human cells, preferably primary human cells. Even more preferably, the cells are immune cells. The cells may be obtained from a subject or from a commercially available culture. The cells can be autologous cells, syngeneic cells, allogenic cells and even in some cases, xenogeneic cells.

In one embodiment, the cells can be selected from a group consisting of a T cell, including CD4⁺ T cell, and C8⁺ T cell, B cell, NK cell, NKT cell, monocyte, granulocyte, e.g., myeloid cells, macrophages, neutrophils, dendritic cells, mast cells, eosinophils, basophils and dendritic cell, preferably the cell being a T lymphocyte, B lymphocyte, natural killer cell, monocyte and antigen presenting cells such as a dendritic cell. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Preferably, the cells are immune cells selected from T lymphocytes, B lymphocytes, natural killer cells, monocytes and antigen presenting cells.

In certain embodiments, the immune cell is a T-cell, e.g., an animal T-cell, a mammalian T-cell, especially a human T-cell. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, α/β T cells, and δ/γ T cells. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPM1-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162).

For instance, suitable immune cells that can be used in the invention include autologous T lymphocyte cells, allogeneic T cells, xenogeneic T cells, progenitors of any of the foregoing, transformed tumor or xenogeneic immunologic effector cells, tumor infiltrating lymphocytes (TIL), cytotoxic lymphocytes or other cells that are capable of killing target cells when activated.

In some other embodiments, the cells are natural killer (NK) cells, Natural Killer T (NKT) cells, cytokine-induced killer (CIK) cells, tumor-infiltrating lymphocytes (TIL), lymphokine-activated killer (LAK) cells, or the like. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT.

Non-limiting exemplary sources for such commercially available cell lines also include the American Type Culture Collection, or ATCC, (http://www.atcc.org/) and the German Collection of Microorganisms and Cell Cultures (https://www.dsmz.de/).

Particularly, the cells used in the method according to the invention are:
Target antigen expressing cells that express the antigen of interest at their surface,
CAR expressing cells that express a CAR, preferably a CAR that has been generated to specifically binds the antigen of interest,
Optionally Control cells that either don't express the antigen of interest or the CAR generated to bind this antigen or both.

Particularly, the target antigen expressing cells and the CAR expressing cell are prepared from the same cell line.

In one embodiment, the method further comprises control cells that do not express the targeted antigen. Particularly, the control cells are cells that does not express the antigen of interest recognized by the CAR expressing cells or express an antigen that is not recognized by the CAR expressing cells. This control cell is a negative control.

In another embodiment, the method further comprises control CAR cells that do not recognize the targeted antigen. Particularly, the control CAR-cells are cells that express a CAR that does not recognize the targeted antigen. This control CAR cell is a negative control.

Particularly, the control cells and/or the control CAR-cells are prepared from the same cell line than the CAR expressing cells and the target antigen expressing cells.

In one embodiment, the antigen expressed by the target antigen expressing cells is HLA-G. “HLA-G” designates the Human leukocyte antigen G which includes at least seven isoforms. Preferably, the antigen expressed by the target antigen expressing cells is an HLA-G isoform selected from HLA-G1, HLA-G2, HLA-G3, HLA-G4, HLA-G5, HLA-G6, and HLA-G7.

The primary transcript of HLA-G is alternatively spliced resulting in the expression of seven isoforms, where four are membrane-bound (HLA-G1, HLA-G2, HLA-G3 and HLA-G4) and three are soluble (HLA-G5, HLA-G6 and HLA-G7). HLA-G1 and HLA-G5 are the most abundant isoforms and they present the typical structure of a classical HLA class I molecule: a heavy chain constituted of three globular domains non-covalently bound to β2-microglobulin (β2M) and a peptide, while the other isoforms are shorter, lacking one or two domains of the heavy chain, and should not bind β2M. HLA-G1 isoform is the complete isoform with α1, α2 and α3 domains associated with β2-microglobulin. The HLA-G2 isoform has no α2 domain, while HLA-G3 has no α2 and α3 domains, and HLA-G4 has no α3 domain. None of the isoforms HLA-G2, HLA-G3 and HLA-G4 binds β2M. The soluble HLA-G5 and HLA-G6 isoforms contain the same extra globular domains than HLA-G1 and HLA-G2, respectively. The HLA-G7 isoform has only the α1 domain linked to two amino acids encoded by intron 2. HLA-G5 isoform binds β2M while the isoforms HLA-G6 and HLA-G7 don't bind β2M.

In a particular embodiment, the antigen of interest expressed by the target antigen expressing cells is a β2M-free HLA-G isoform.

In one embodiment, the CAR expressed by the CAR expressing cells is a CAR that specifically binds some HLA-G isoform(s), preferably two to five, more preferably selected from HLA-G1, HLA-G2, HLA-G5 and HLA-G6. Preferably, the CAR expressed by the CAR expressing cells is a CAR that specifically binds to both HLA-G1 and HLA-G5 or to both HLA-G2 and HLA-G6. Alternatively, the CAR expressed by the CAR expressing cells is a CAR that specifically binds to both HLA-G2 and HLA-G6. Optionally, the CAR expressed by the CAR expressing cells is a CAR that specifically binds to either β2M-free HLA-G or β2M-associated HLA-G.

Cells Labelling

According to the method of the invention, the target antigen expressing cells and CAR expressing cells are labelled with different labels. Despite only one label could be used in the method, the use of different labels for target antigen expressing cells and CAR expressing cells provides a higher efficiency to the method.

Then, preferably, the target antigen expressing cells and/or the control cells are labeled with a marker that can be distinguishable from the marker used to label the CAR expressing and/or the control CAR cells.

According to the invention, the amount of marker is sufficient to label the cell to make it detectable and/or traceable. Such amounts are known to the person skilled in the art and depend on the choice of a suitable detection system as well as the choice of a suitable amount of marker, which is well within the skill of the person skilled in the art.

Preferably, the label is non-toxic and/or non-mutagenic and can be detected in a living cell. However, an advantage of the present invention is that also a mutagenic and/or slightly toxic markers can be used, as the labeling is performed in vitro. The label that can be used according to the invention include radioactive label and non-radioactive label. Non-radioactive labels include optically detectable labels, such as for example fluorescent, luminescent and phosphorescent labels or dyes, preferably fluorescent ones. Labels include directly detectable and indirectly detectable labels. In one embodiment, the label is a fluorescent label, preferably a label suitable for flow cytometry analysis.

Particularly, the label of the invention is a membrane marker that can be incorporated into the cell membrane such as lipid markers or dyes. Preferred fluorescent molecules chosen to be incorporated into the cell membrane are MINI26, PKH26-PCL, PKH67, MINI67, PKH67-PCL, PKH26, PKH26-PCL, Vybrant CM-Dil, Dil and DiO. In one embodiment, the marker used to label the cells is selected from the group consisting of PKH67 and PKH26.

When control cells are used, control cells that do not express the targeted antigen can be labelled with the same label than the target antigen expressing cells or with a different label. Similarly, when control CAR-cells are used, control CAR-cells that do not recognize the targeted antigen can be labelled with the same marker than the CAR expressing cells or with a different label.

In one embodiment, antigen expressing cells and/or control cells are labelled with PKH67 and CAR expressing cells and/or control CAR-cells are labelled with PKH26 or vice versa.

Labeling of cells allows to investigate the functionality of the CAR expressing cells and optionally to isolate the functional CAR expressing cells (e.g. by fluorescence-activated cell sorting, FACS).

Any method known by the person skilled in the art can be used to label cells, particularly any method known in the art to label the membrane of cells

Particularly, the labelling of target antigen expressing cells and CAR expressing cells and/or control cells with different labels comprises:

a) providing a cell or a cell culture, particularly a target antigen expressing cell culture and/or a CAR expressing cell culture and/or a control cell and/or a control CAR-cell,

b) contacting the culture or cells with a label, particularly a membrane marker, preferably selected from MINI26, PKH26-PCL, PKH67, MINI67, PKH67-PCL, PKH26 and PKH26-PCL,

c) optionally, washing the culture in order to remove excess label,

d) optionally, repeating steps b) and c),

e) optionally further cultivating the cell or culture, preferably for at least 1 day, at least 2 days, more preferably at least 4 days, even more preferably at least 1 week or at least 2 weeks

g) optionally selecting and/or collecting the labeled cells.

Thus, in some embodiments, the cell populations may be incubated in a culture composition prior to the co-incubation.

Co-Incubation

After labelling, the target antigen expressing cells, the CAR expressing cells, and optionally the control cells, are co-incubated.

In one embodiment, the CAR expressing cells are co-incubated with antigen expressing cells and/or with control cells. When the co-incubation comprises either the CAR expressing cells with the antigen expressing cells or the CAR expressing cells with control cells separately, control cells and antigen expressing cells can be labelled with the same marker. When the co-incubation comprises the CAR expressing cells with the antigen expressing cells and the control cells, each of these three populations are labelled with different markers.

In another embodiment, the antigen expressing cells are co-incubated with CAR expressing cells and/or with control CAR-cells. When the co-incubation comprises either the antigen expressing cells with the CAR expressing cells or the antigen expressing cells with control CAR-cells separately, control CAR-cells and CAR expressing cells can be labelled with the same marker. When the co-incubation comprises the antigen expressing cells with the CAR expressing cells and the control CAR-cells, each of these three population is labelled with a different marker.

When the co-incubation comprises CAR expressing cells, control CAR-cells, antigen expressing cells and control cells, each of these four population is labelled with a different marker.

The cells can be incubated by any method known by the person skilled in the art, depending on the particular conditions needed by the cells of the invention. The conditions can include one or more of particular media, temperature, oxygen content, carbon dioxide content, time, agents, e.g., nutrients, amino acids, antibiotics and/or ions.

In some embodiments, the incubating conditions include temperature suitable for the growth of human immune cells (e.g. T cells), for example, at least about 25° C., generally at least about 30° C., and generally at or about 37° C.

The incubation may be carried out in a culture vessel, such as a unit, chamber, well, column, tube, tubing set, valve, vial, culture dish, bag, or other container for culture or cultivating cells.

Preferably, the co-incubation is performed at least 1 hour, at least 2 hours, at least 3 hours, at least 4 hours or at least 5 hours, preferably during 1 to 5 hours, even more preferably during 1 to 3 hours.

In one embodiment, the co-incubation is performed in a 1:0.5 to a 1:10 target antigen expressing cells to CAR expressing cells ratio, preferably in a 1:1 to a 1:10 target antigen expressing cells to CAR expressing cells ratio. Preferably, cells concentration prior to incubation is at least 10⁴ cells/mL, at least 10⁵ cells/mL, or at least 10⁶ cells/mL preferably at least 10⁶ cells/mL, even more preferably 2×10⁶ cells/mL.

In the context of the invention, the co-incubation of cells leads to the contact of cells, particularly of two or more populations of cells such as the CAR-cell and the antigen expressing cells, with or without the presence of control cells and/or control CAR-cells.

In a particular aspect, the method further comprises a step of incubating target antigen expressing cells with an antibody, preferably an antibody that recognizes the same or an overlapping epitope than the antibody from which the CAR is derived, prior to co-incubating the target antigen expressing cells with the CAR expressing cells. Preferably, the antibody is a monoclonal antibody that comprises at least one of the CDR of the monoclonal antibody from which the CAR is derived, preferably 2, 3, 4, 5 or 6 CDRs of the monoclonal antibody from which the CAR is derived. This particular aspect allows to test the CAR specificity, i.e., if the CAT activation is made through the binging of the antigen of target antigen cell by the CAR of the CAR expressing cells. Indeed, if the CAR activation (and then membrane transfer) is inhibited or decreased in presence of the antibody, it can be concluded that the effect if specific of the CAR effect on antigen and that the CAR expressing cells are specific to the targeted antigen. A negative control can be added by carry out the same test but with an antibody which is not able to bind the antigen.

Membrane Acquisition Analysis and Cells Sorting

Trogocytosis involves transfer of plasma membrane and anchored proteins from either the APC or the tumor cells to the effector immune cells (e.g. CAR expressing cells). Trogocytosis is dependent on cell-to-cell contact. Indeed, trogocytosis is an active process which broadly reflects the antigen-specific activation of the effector cells. During this conjugation, membrane transfers are dependent on the activation state of the acquirer cells: trogocytosis requires CAR expressing cells activation in the presence of cells harboring the antigen they specifically recognize. Thus, the extent of trogocytosis is related to the activation of functionally immune cells such as B, αβ T, γδ T, and NK effector cells, triggered by antigen receptor signaling on T and B cells, by killer inhibitory and killer activatory receptor on NK cells. Yet, membrane transfers are not only related to activation state but also to function since the T cells performing trogocytosis also expose at their surface a marker of their cytotoxic function: CD107a. Thus, a method based on trogocytosis can make membrane transfer an original and fast method to investigate effector cells' specificity and function, particularly when applied to CAR expressing cells.

The method according to the invention comprises a step of analyzing the cells in order to assess membrane acquisition by the CAR expressing cells from the target antigen expressing cells, the membrane acquisition being indicative of the binding and/or activation capacity of the CAR expressing cells to the target antigen, thereby assessing the functionality of the CAR expressing cells.

This membrane transfer can be monitored thanks to the different marker with which the cells have been previously labelled by any method known by the person skilled in the art, according to the label which has been used for cells labeling.

Particularly, when the label is a fluorescent label, membrane transfer can be analyzed by fluorescence microscopy (digital imaging can be used in addition to provide superior resolution and accurate quantitative determinations) or by Fluorescent Activated Cell Sorting (FACS).

FACS system based on flow cytometry is well known in the art and is capable of separating and isolating a desirable cell population from a sample, such as CAR expressing cells that have acquired membrane from target antigen expressing cells. Indeed, when such membrane transfer occurs, the CAR expressing cells contain the two labels with whom the CAR-expressing cells and the antigen expressing cells have been marked with.

Flow cytometry utilizes a fluid stream to linearly segregate particles such that they can pass, single file, through a detection apparatus. Individual cells can be distinguished according to their location in the fluid stream and the presence of detectable markers. Thus, flow cytometry can be used to produce a diagnostic profile of a population of cells, and allows the distinction of different categories of cells, such as antigen expressing cells (e.g. via label 1), CAR expressing cells that did not acquire target cell membrane (e.g. via label 2), functional CAR expressing cells that did acquire target cell membrane (e.g. via both label 1 and label 2), optionally control cells (e.g. via label 1, 2 or 3) and cells that do not express any label.

In one embodiment, detecting the cells in a flow cytometer may include exciting a fluorescent dye with one or more lasers at an interrogation point of the flow cytometer, and subsequently detecting fluorescence emission from the dye using one or more optical detectors. It may be desirable, in addition to detecting the particle, to determine the number of particles (e.g., cells) sorted or separated. Accordingly, in some embodiments, the methods further include counting and/or sorting the labeled particle (e.g. CAR expressing cells that acquired membrane from the antigen expressing cells). In detecting, counting and/or sorting particles, a liquid medium including the particles is first introduced into the flow path of the flow cytometer. When in the flow path, the particles are passed substantially one at a time through one or more sensing regions (e.g., an interrogation point), where each of the cells is exposed individually to a source of light at a single wavelength and measurements of light scatter parameters and/or fluorescent emissions as desired (e.g., two or more light scatter parameters and measurements of one or more fluorescent emissions) are separately recorded for each particle. The data recorded for each particle is analyzed in real time or stored in a data storage and analysis means, such as a computer, as desired. U.S. Pat. No. 4,284,412 describes the configuration and use of a flow cytometer of interest equipped with a single light source while U.S. Pat. No. 4,727,020 describes the configuration and use of a flow cytometer equipped with two light sources. Flow cytometers having more than two light sources may also be employed. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 535 nm, 635 nm, and the like. Accordingly, in flow cytometrically assaying the cells, the cells may be detected and uniquely identified by exposing the cells to excitation light and measuring the fluorescence of each particle in one or more detection channels, as desired. The excitation light may be from one or more light sources and may be either narrow or broadband.

In series with a sensing region, detectors, e.g., light collectors, such as photomultiplier tubes (or “PMT”), are used to record light that passes through each particle (in certain cases referred to as forward light scatter), light that is reflected orthogonal to the direction of the flow of the cells through the sensing region (in some cases referred to as orthogonal or side light scatter) and fluorescent light emitted from the cells, if it is labeled with fluorescent marker(s), as the particle passes through the sensing region and is illuminated by the energy source. Each of forward light scatter (or FSC), orthogonal light scatter (SSC), and fluorescence emissions (FL1, FL2, etc.) include a separate parameter for each particle (or each “event”).

Flow cytometers of interest that may be used for the purpose of the invention may include BD Biosciences FACSCanto™ flow cytometer, BD Biosciences FACSVantage™, BD Biosciences FACSort™, BD Biosciences FACSCount™, BD Biosciences FACScan™, BD Accuri C6 Plus™, Miltenyi MacsQuant™, or the like.

In one embodiment, membrane transfer is monitored by flow cytometry analysis in accordance to the use of PKH67 and PKH26 labels.

In the embodiment where the antigen expressing cells are labelled with PKH67 and the CAR expressing cells are labelled with PKH26 or vice versa, membrane transfer analysis is performed by FACS. In this embodiment, it is expected that membrane transfer occurs so that cells may be sorted in at least three categories: (i) cells expressing only PKH67, (ii) cells expressing only PKH26, (iii) cells expressing both PKH67 and PKH26 and (iv) eventually cells that neither express PKH26 nor PKH67. Accordingly, the percentage of cells expressing both of the PKH26 and the PKH67 labels may be established to monitor the percentage of CAR expressing cells that effectively acquire cell membrane from the antigen expressing cell, thereby monitoring the antigen-CAR interaction and the functionality of the CAR expressing cells.

In the embodiment where the method further comprises control cells or control CAR-cells, control cells or control CAR-cells can be sorted by flow cytometry via the label PKH67, PKH26, Dil, Vybrant CM-Dil, DiO or other lipophilic dyes, or commercially available lipophilic dyes.

Particularly, in the embodiment comprising control cells, where the control cells are labelled with PKH67 and the CAR expressing cells are labelled with PKH26 or vice versa, membrane transfer analysis is performed by FACS. In this embodiment, it is expected that no membrane transfer occurs so that cells may be sorted in at least two categories: (i) cells expressing only PKH67, (ii) cells expressing only PKH26, (iii) eventually cells that neither express PKH26 nor PKH67.

In the embodiment comprising control CAR-cells, where the control CAR-cells are labelled with PKH67 and the antigen expressing cells are labelled with PKH26 or vice versa, membrane transfer analysis is performed by FACS. In this embodiment, it is expected that no membrane transfer occurs so that cells may be sorted in at least two categories: (i) cells expressing only PKH67, (ii) cells expressing only PKH26, (iii) eventually cells that neither express PKH26 nor PKH67.

In a particular embodiment, the method further comprises the selection of the functional CAR expressing cells and/or the functional CAR construct. Indeed, this method allows the selection of cells that express a particular CAR construct that was tested to be functional toward the antigen of interest. By data analysis, for example of the percentage of membrane transfer occurring, this method allow the selection of the most suitable CAR expressing cells and/or CAR construct towards an antigen of interest. This method thus also allows the selection of the most suitable CAR components (e.g. transmembrane domain, hinge, intracellular domains) that are known to affect CAR expressing cells functionality. This particular selection of functional CAR expressing cells and/or the functional CAR construct is of great interest to improve CAR-based therapies.

Method for Preparing Functional CAR Expressing Cells for Adoptive Cell Therapy

Methods of introducing genes into a cell and expressing genes in a cell are known in the art.

Particularly, methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In the context of an expression vector, the vector can be readily introduced into a host cell, e.g., mammalian, bacterial, yeast, or insect cell by any method in the art. For example, the expression vector can be transferred into a host cell by physical, chemical, or biological means that are more particularly described here below.

In some embodiments, the methods include isolating cells from the subject, preparing, processing, culturing, and/or engineering them, as described herein, and re-introducing them into the same patient, before or after cryopreservation.

Here is particularly provided a method of producing CAR expressing cells comprising, or alternatively consisting essentially of, or yet further consisting of the steps: (i) transducing a population of isolated cells with a nucleic acid sequence encoding a CAR; and (ii) selecting a subpopulation of said isolated cells that have been successfully transduced with said nucleic acid sequence of step (i) thereby producing CAR expressing cells. In one aspect, the isolated cells are selected from a group consisting of T-cells and NK-cells.

Here is even more particularly provided a method of producing CAR expressing cells comprising, or alternatively consisting essentially of, or yet further consisting of the steps:

(i) transducing the population of immune cells with a nucleic acid sequence encoding a CAR, preferably wherein the population of immune cells has been isolated from a biological sample from a subject to be treated,

(ii) selecting a subpopulation of said isolated cells expressing the CAR and,

(iii) assessing their functionality by the method according to any of the preceding claims, preferably towards target antigen expressing cells that expresses the antigen for which the CAR has been generated

(iv) optionally selecting the functional CAR expressing cells and/or the CAR nucleic acid construct from the CAR expressing cells that experienced the membrane acquisition form the antigen expressing cells.

In a particular embodiment, the method further comprises:

(i) providing immune cells populations, preferably from a biological sample from a subject to be treated (e.g. blood cells),

(ii) isolating a particular immune cell population (e.g. T cells, B lymphocytes, monocyte and antigen presenting cells and/or NK cells) prior to the transduction of the population of immune cells with a nucleic acid sequence encoding a CAR.

In another embodiment, the method further comprises selecting the CAR expressing cells and/or the CAR construct from the CAR expressing cells that experienced the membrane acquisition form the antigen expressing cells. Subsequently, the transduced immune cells construct that correspond to the CAR expressing cells that experienced the membrane acquisition from the antigen expressing cells can be further reintroduced or administered to the subject. This method allows the selection of the most suitable CAR expressing cell to target an antigen of interest by assessing its functionality prior to any introduction of such engineered cell into a subject in need thereof.

To facilitate administration, the transduced cells according to the invention can be made into a pharmaceutical composition or made into an implant appropriate for administration in vivo, with pharmaceutically acceptable carriers or diluents by any method known in the art to be suitable for administration to a subject.

Once the cells expressing the CAR construct that has been selected from the method according to the invention are administered to a subject, the biological activity of the engineered cell populations and/or antibodies in some aspects is measured by any of a number of known methods. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as GM-CSF, IL-3, MIP-la, TNF-α, IL-10, IL-13, IFN-γ, or IL-2.

In some aspects the biological activity is measured by assessing clinical outcome, such as progression free survival, or overall survival, reduction in tumor burden or load, stabilization of tumor.

### Examples—Results

Membrane Acquisition Through CAR/HLA-G1 Interaction

It was previously demonstrated that immune cells acquire cell surface membrane patches from target cells such as APC or tumor cells antigen-presenting cell (Caumartin et al., EMBO J, 2007. 26(5): p. 1423-33). Membrane acquisition from target cells by effector cells (i) require cell-to-cell contact, (ii) is rapid, and (iii) an active process dependent on the activation state of the acquirer cells which (iv) reflects the antigen-specific activation of the effector cells. Therefore, membrane transfers are related to the activation of functionally mature immune cells.

It was investigated if, following CAR engagement, effector cells could be activated and then acquire membrane patches from target cells. For this purpose, experiments were set up using the Jurkat T cell line as effector as well as target cells to avoid any other bystander protein-protein interactions than HLA-G/CAR interaction. This implies that cell-to-cell contact would only be mediated/driven by the CAR—antigen interaction.

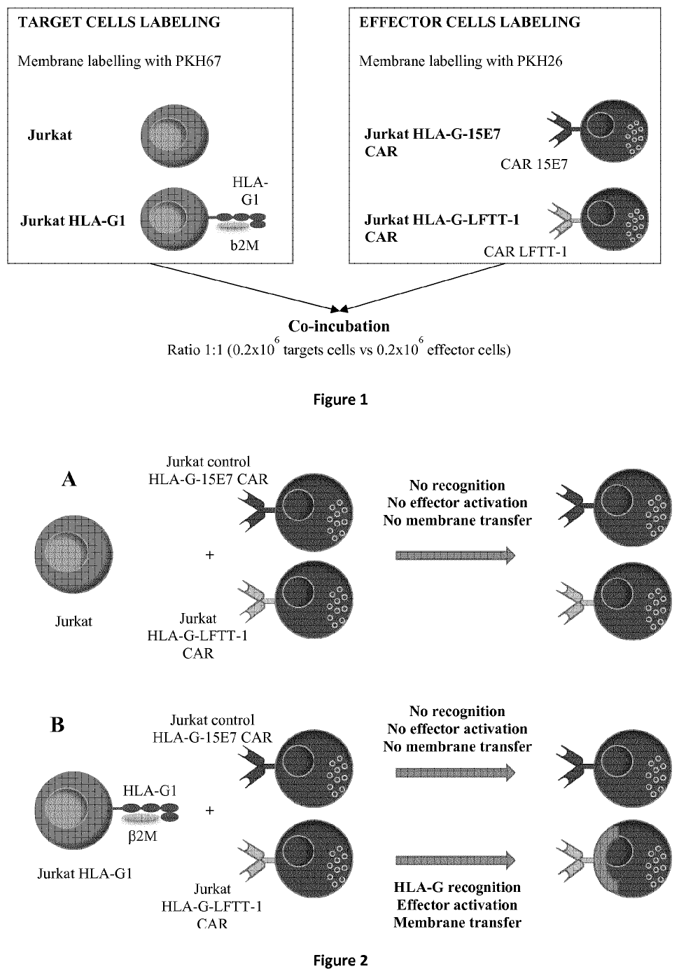

As shown on FIG. 1, membranes of Jurkat target cells were labeled in green using the PKH67 fluorescent dye whereas Jurkat effector CAR cell membranes were labeled in red using the PKH26 fluorescent dye. To determine whether membrane transfers were related to the CAR—HLA-G interaction, we first compared the extent of membrane transfers between HLA-G expressing target cells and CAR effector cells. For this purpose, two different HLA-G CAR constructs were generated depending on their binding to HLA-G1 isoform: the HLA-G CAR based on LFTT-1 antibody paratope is specific for HLA-G1 isoform associated to β2M whereas the “control HLA-G CAR” based on 15E7 antibody paratope is only specific for HLA-G1 isoform non associated to β2M. Jurkat HLA-G1 cell lines only expressed HLA-G1 associated to β2M isoform which is only recognized by LFTT-1 antibody and not the 15E7. As represented on FIG. 2A, in absence of HLA-G expression on target cells, no membrane acquisition from target cells is expected by effector CAR cells. However, on FIG. 2B, in presence of HLA-G expressing target cells, Jurkat cells expressing the specific HLA-G-LFTT-1 CAR should acquire membrane patches from HLA-G1/β2M associated target cells whereas Jurkat cells expressing the control HLA-G-15E7 CAR should not.

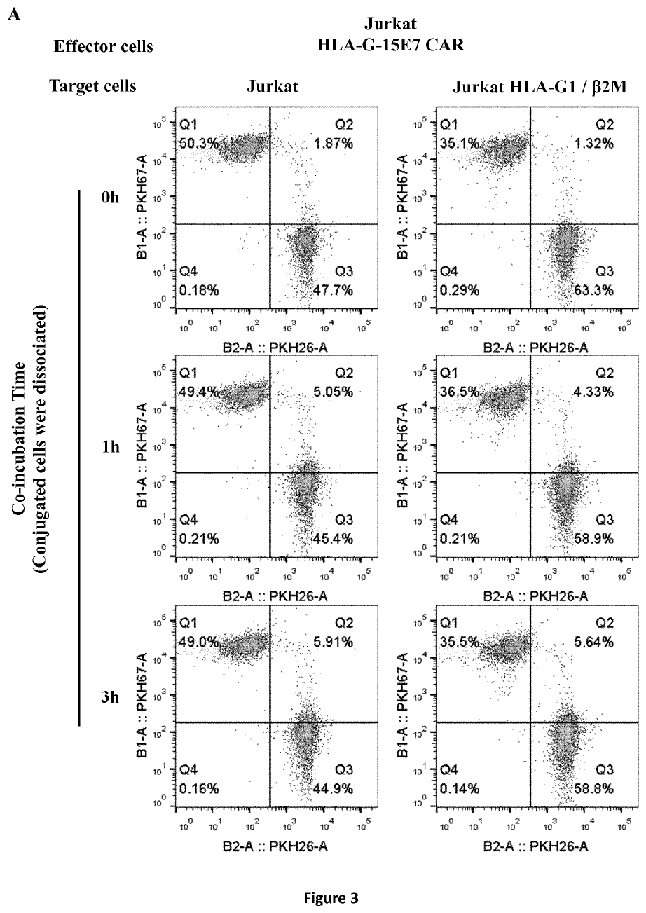

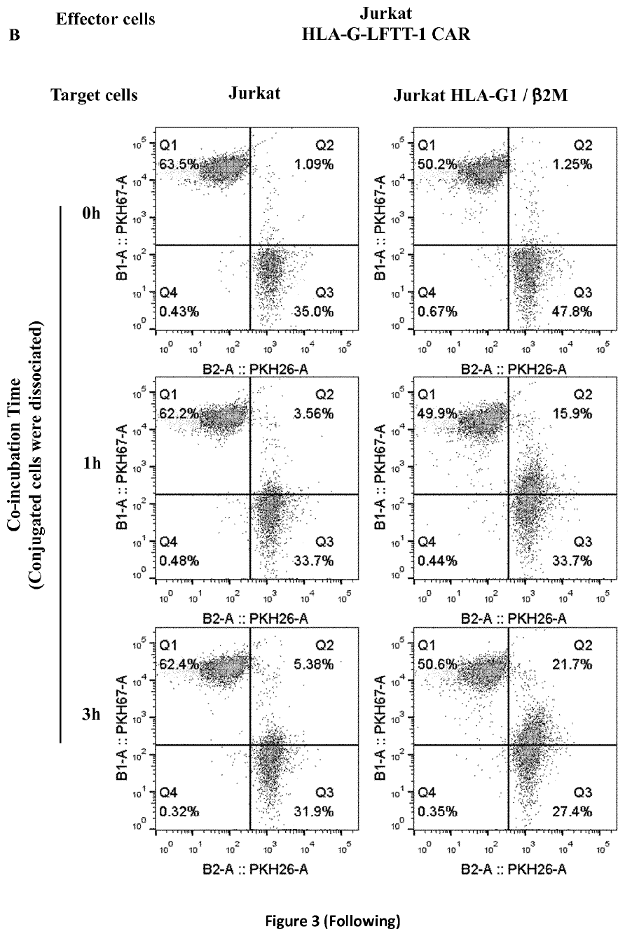

Target and effector cells were coincubated during 1 and 3 hours and membrane transfers were then analyzed by flow-cytometry. As shown on FIGS. 3A and 3B, prior to coincubation, CAR effector cells did not acquire membrane from tumor target cells either expressing or not HLA-G. After 1 hour coincubation, CAR effector cells not specific for HLA-G1/β2M proteins did not acquire membrane patches neither from HLA-G1 transduced nor HLA-G1 negative tumor cells. However, 15.9% of HLA-G1/β2M specific CAR cells already acquire membrane patches from HLA-G1 tumor cells but not from their HLA-G1 negative counterparts.

Altogether, membrane transfer was demonstrated between HLA-G1/β2M expressing tumor cells and CAR effector cells specific for HLA-G1/β2M antigen.

Membrane Acquisition is a Rapid Process

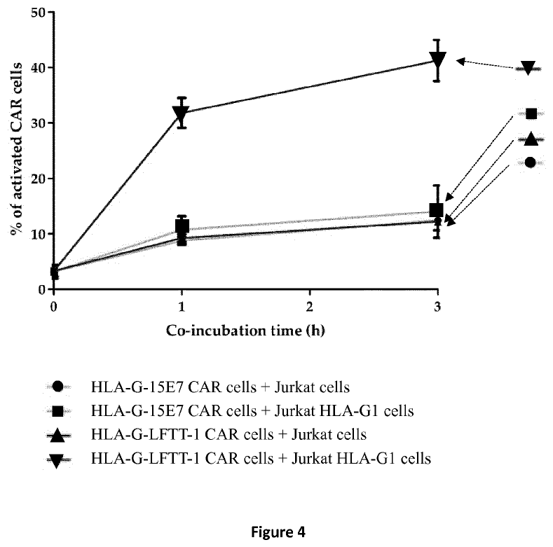

Membrane transfer between HLA-G1/β2M tumor cells and HLA-G CAR effector cells was confirmed after a 3 hours coincubation where almost 22% of LFTT-1 CAR effector cells were capable to perform trogocytosis. Yet, 15E7 CAR cells were not capable to acquire membrane from HLA-G1/β2M target cells even after a 3 hour coincubation. As shown on FIG. 4, kinetic of membrane acquisition is rapid since membrane transfers concern +/−32% of HLA-G-LFTT-1 CAR effector cells after a 1 h coincubation, and 3 hours of coincubation slightly increased the proportion of effector cells capable of trogocytosis to +/−40%.

These results showed that membrane transfers are fast and only related to the antigen/CAR specific interaction since HLA-G-LFTT-1 CAR effector cells only acquired membrane fragments from HLA-G1/β2M positive tumor cells whereas HLA-G-15E7 CAR effector cells did not acquire membrane patches from HLA-G1/β2M positive tumor cells.

Membrane Acquisition is Dependent on CAR Specificity

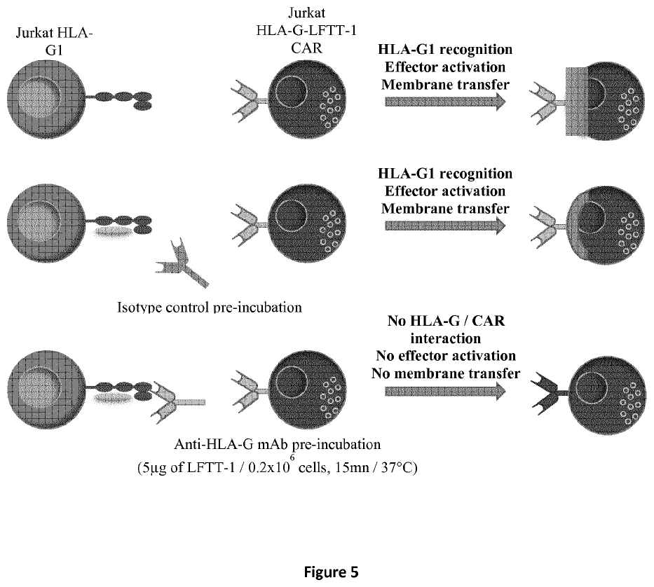

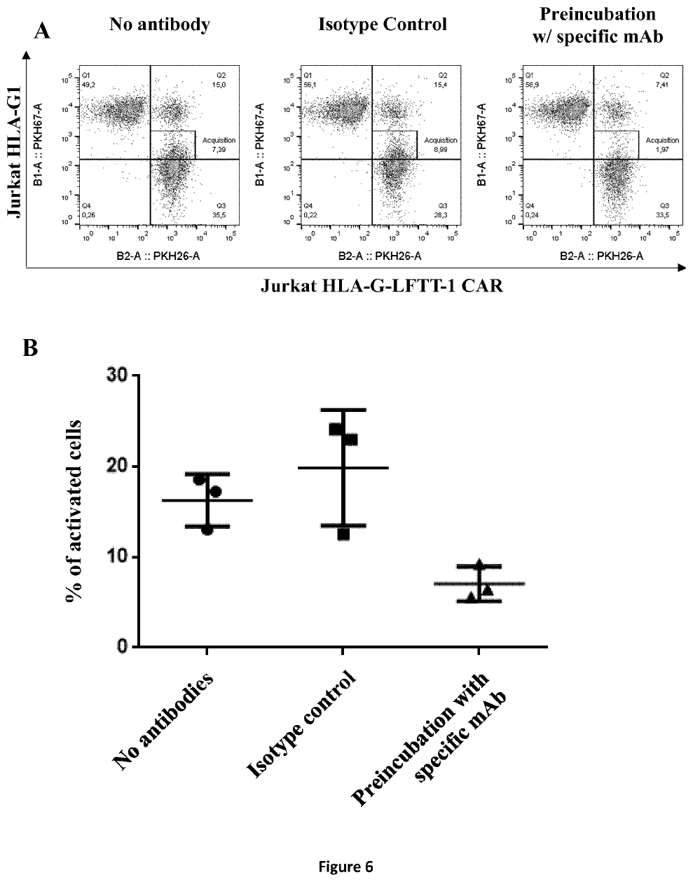

Then, the relation between trogocytosis and CAR specificity was assessed. To do so, as schematized on FIG. 5, prior to trogocytosis experiments, HLA-G1 transduced tumor cells were incubated either with the LFTT-1 antibody, which paratopes was used to generate the HLA-G-LFTT-1 CAR construct, or with its isotype control antibody. HLA-G1 CAR effector cells were then incubated 1 hour with these tumor cells and membrane acquisition was investigated by flow-cytometry (FIG. 6A). Pre-incubation with the isotype control antibody did not prevent HLA-G1 CAR effector cells to perform trogocytosis (FIG. 6B). Yet, LFTT-1 pre-incubation almost completely abrogated the trogocytosis process.

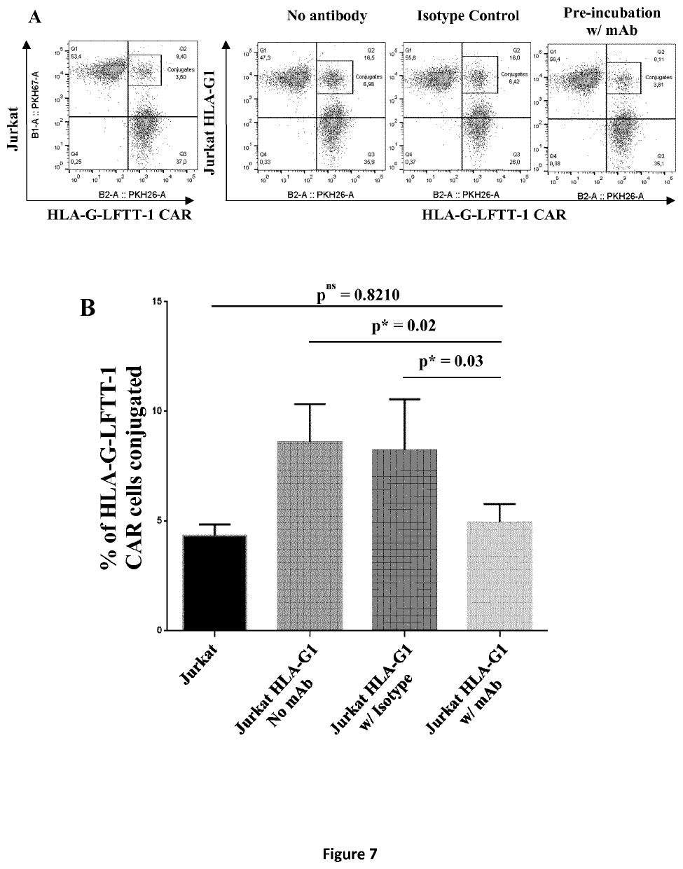

Trogocytosis is dependent on cell-to-cell contact between target cells and effector cells mediated by the antigen—CAR interaction. For this purpose, it was investigated whether pre-incubation with LFTT-1 antibody could inhibit conjugates formation between HLA-G1 tumor cells and HLA-G-LFTT-1 CAR cells (FIG. 7A). It was determined that trogocytosis was inhibited because pre-incubation with LFTT-1 antibody blocked the interaction between the target cells and CAR effector cells. Indeed, HLA-G1 CAR effector cells were no longer capable to establish cell-to-cell contact with HLA-G1 tumor cells and were no longer capable to acquire membrane (FIG. 7B).

Altogether, trogocytosis is a potent method to determine CAR characteristics since membrane transfers are (i) specific, (ii) mediated by CAR/antigen interaction, cell-to-cell contact dependent and (iv) rapid. Trogocytosis from HLA-G1/β2M expressing target cells only by HLA-G-LFTT-1, and not by HLA-G-15E7 CAR effector cells demonstrated that trogocytosis is particularly relevant to determine CAR specificity.

Membrane Transfers are Specific to CAR Interacting Cells

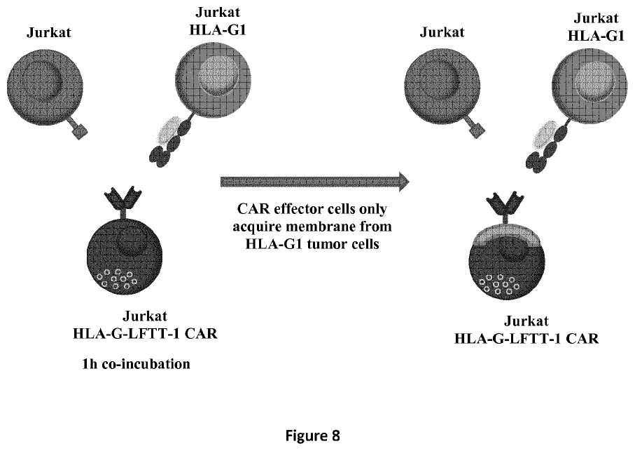

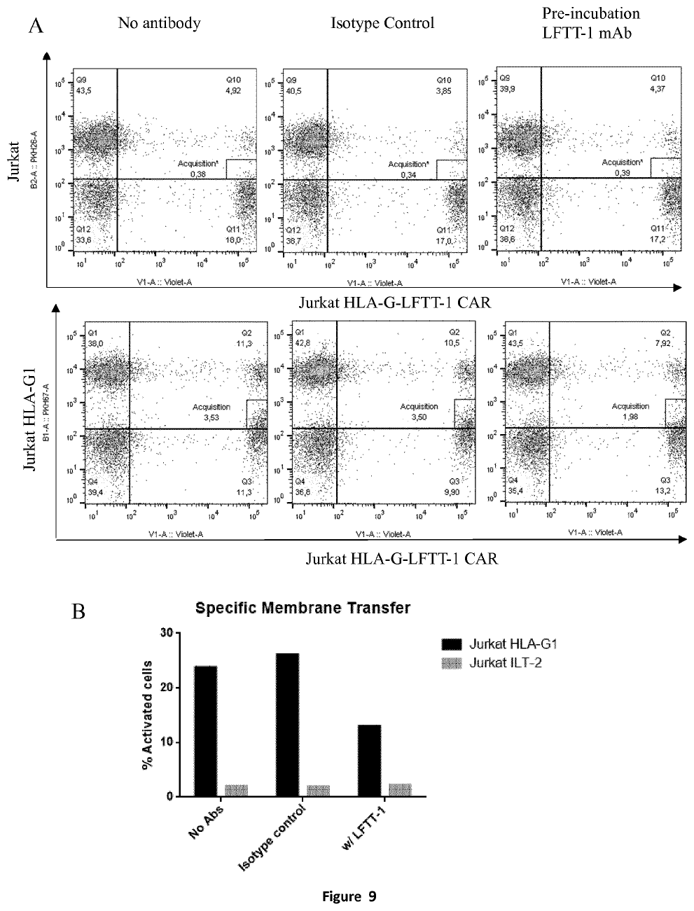

Following CAR stimulation, it was further investigated whether CAR activated effector cells only interact with antigen specific expressing tumor cells or if they could have bystander effects. To do so, HLA-G1 negative tumor cells labeled in red with HLA-G1 positive tumor cells labeled in green and HLA-G-LFTT-1 CAR effector cells labeled in blue (FIG. 8) were co-incubated during 1 hour. Membrane acquisition by CAR effector cells were then analyzed by flow-cytometry. As shown on FIG. 9, after co-incubation, HLA-G-LFTT-1 CAR effector cells only acquired green membrane patches demonstrating that, activated HLA-G-LFTT-1 CAR effector cells only interact with HLA-G1 expressing tumor cells and not with bystander cells.

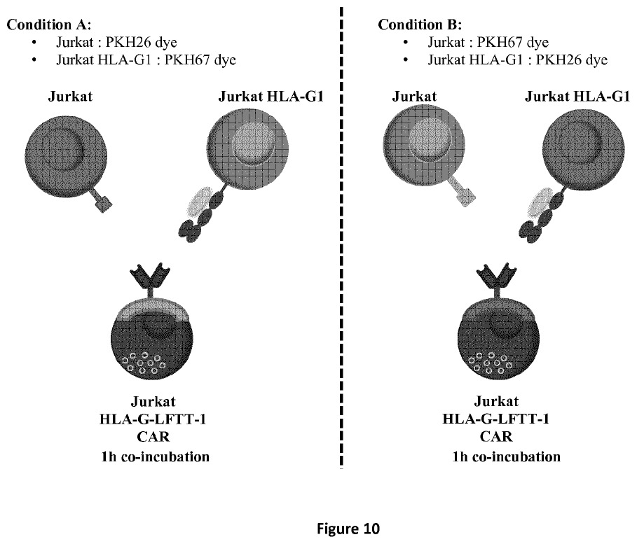

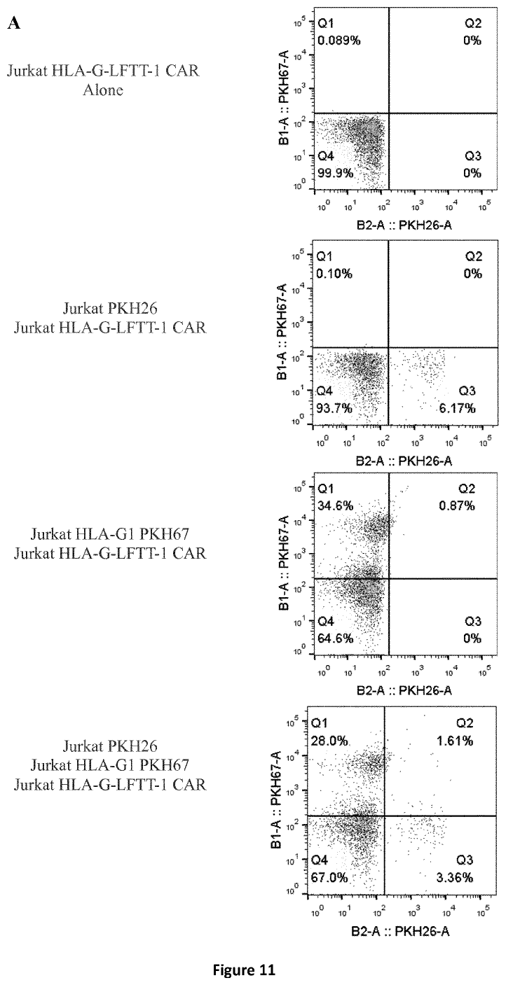

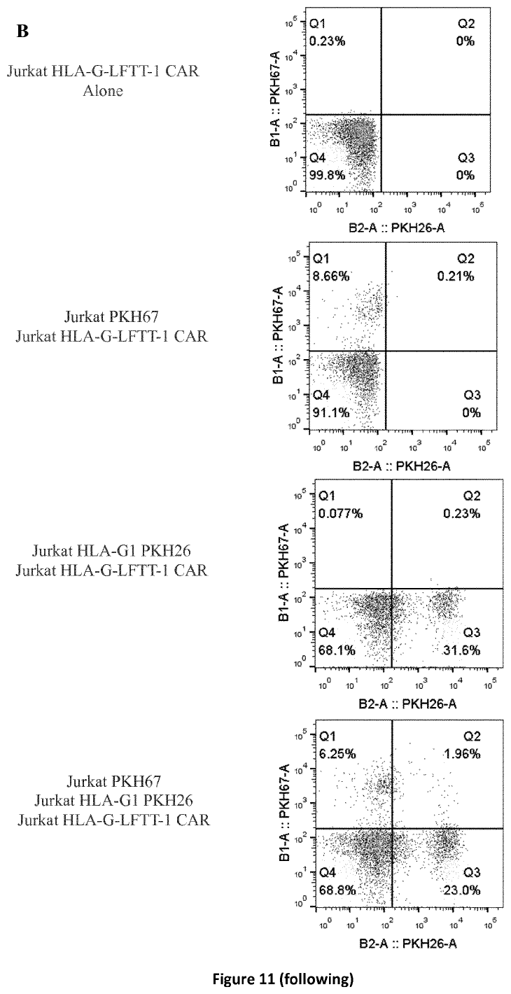

The membrane transfer observed was not influenced by the dye used to identify the target cells. As shown on FIGS. 10, 11A and 11B, HLA-G-LFTT-1 CAR effector cells acquired red or green membrane dye to the same extent from HLA-G1 expressing tumor cells showing that trogocytosis was not dependent on the membrane dye used.

### Material and Methods

Cells Lines

Jurkat cell line is human CD4+ T cells purchased from the ATCC (American Type Culture Collection TIB-152). Jurkat cell line was transduced with HLA-G1, HLA-G-LFTT-1 or HLA-G-15E7 CAR lentivirus, respectively. Jurkat wt and Jurkat transduced cell lines were cultured in RPMI 1640 (Invitrogen) supplemented with 2 mM L-glutamine, 1 mg/ml penicillin and streptomycin (X), and 10% heat-inactivated FCS (Invitrogen).

Jeg-3 cell line are human choriocarcinoma cells purchased from the ATCC (American Type Culture Collection HTB-36). These were cultured in MEM (X) supplemented with 1 mg/ml penicillin and streptomycin (X), and 10% heat-inactivated FCS (Invitrogen).

CAR-HLA-G Jurkat Cells

HLA-G-LFTT-1 and HLA-G-15E7 CAR constructs were generated as previously described [7]. Briefly, the anti-HLA-G-recognizing domain is a single-chain variable fragment (scFv) derived from HLA-G1/β2M associated specific antibody LFTT1 (REF Patent CAR HLA-G) or from the HLA-G1/β2M free specific antibody 15E7 (REF Patent). A short spacer derived from the IgG1 hinge region was used to link this scFv to the transmembrane domain. The HLA-G CAR endodomain was constituted by the fusion of CD28, OX40 and CD3z activation molecules. CAR construct was cloned into a pTrip plasmid vector by digestion/ligation after extraction by PCR with specific primers, under CMV immediate early promoter.

HLA-G Jurkat Cells

HLA-G-expressing stable Jurkat cell lines was generated by transduction and the lentiviral particles were generated as follows: specific sequences corresponding to native HLA-G1 cDNA (NM_002127.5) modified K334A and K335A according to Longmei Zhao et al. [38] were cloned separately into a pTrip plasmid vector by digestion/ligation after extraction by PCR with specific primers, under CMV immediate early promoter.

Lentiviral Vectors

HIV-1-derived vector particles were produced by calcium phosphate co-transfection of HEK-293T cells (ATCC) with the recombinant plasmid pTRIP, an envelope expression plasmid encoding the glycoprotein from VSV, serotype Indiana glycoprotein, and the p8.74 encapsidation plasmid. Viral stocks were titrated by real-time PCR on cell lysates from transduced HEK-293T cells and expressed as transduction unit (TU) per ml.

To generate Jurkat HLA-G-LFTT-1, HLA-G-15E7 CAR cells and Jurkat HLA-G, 1×10⁵ Jurkat cells were seeded in 12-well plate with in 500 μl of cRPMI medium and 10⁶TU (293T) of Trip CMV-CAR-HLA-G-LFTT-1, Trip CMV-CAR-HLA-G-15E7 or Trip CMV-HLA-G vectors respectively. Cells were incubated for 1 hour at 37° C. and then centrifuged 1 hour at 37° C. 1200 g. Afterwards, 1 ml of cRPMI medium was added and incubated at 37° C. Two weeks later, positive cells were sorted by flow cytometry using anti-HLA-G antibodies. The expression of HLA-G was evaluated by flow cytometry before the Jurkat HLA-G CAR activation assay.

Trogocytosis Experiments

Jurkat HLA-G CAR effector cells and either Jurkat or Jurkat HLA-G1 tumor cells were respectively labeled with PKH26 and PKH67 fluorescent dyes (Sigma) according to the manufacturer's specifications.

For trogocytosis assays, Jurkat HLA-G-LFTT-1 or HLA-G-15E7 CAR (“acquirer” cells) were co-cultured with either Jurkat or Jurkat HLA-G1 cells (“donor” cells) for 1 h at a 1:1 effector-tumor ratio, in a total concentration of 2×10⁶ cells/mL, and at 37° C. in a 5% CO₂ humidified incubator (Lemaoult et al. 2007 Blood J; Caumartin et al. 2007 EMBO J). At the end of the co-incubation, cells were placed on ice and all further steps were performed at less than 4° C. Acquisition of tumor cell membrane by CAR effector cells was investigated by flow cytometry. When indicated, conjugated cells were dissociated through vortexing before FACS acquisition.

Flow Cytometry Analysis

The analyses were carried out using an BD LSR FORTESSA equipped with Diva software (BD Bioscience), under the following experimental conditions: cells in suspension in a PBS isotonic buffer at pH 7.4, having an osmolality of 320-330 mOsmol/kg, with the number of cells analyzed being 10,000.

CAR Blocking Procedures

For blocking HLA-G/CAR interactions, Jurkat HLA-G tumor cells were pre-incubated with 5 μg/ml blocking LFTT-1 antibody or its IgG1 isotypic control prior co-incubation experiments with Jurkat HLA-G-LFTT-1 CAR effector cells.

Statistical Analyses

Data are presented as means+/−standard deviation (SD). Student t test was used and a P value less than 0.05 was taken to be significant. For figures showing representative experiments, error bars represent SD of triplicates.

### CONCLUSION

Here is reported a new method based on membrane transfer between target cells and activated effector cells to determine CAR functionality. Furthermore, membrane acquisition extent is directly correlated to the activation state of the effector cells.

Based on trogocytosis parameters, this method is an original assay to rapidly determine and evaluate CAR specificity, functionality and sensitivity.

1. An in vitro method for assessing the functionality of chimeric antigen receptor (CAR) expressing cells, comprising:
Labelling target antigen expressing cells and CAR expressing cells with different labels, wherein the target antigen expressing cells and the CAR expressing cells are prepared from the same cell line;
Co-incubating the labelled target cells and the labelled CAR expressing cells,
Analyzing the cells in order to assess membrane acquisition by the CAR expressing cells from the target antigen expressing cells, the membrane acquisition being indicative of the binding and/or activation capacity of the CAR expressing cells to the target antigen, thereby assessing the functionality of the CAR expressing cells; and wherein the cells are immune cells.

2. The method of claim 1, wherein the co-incubation is performed at least 1 hour, preferably during 1 to 5 hours, even more preferably during 1 to 3 hours.

3. The method of claim 1 or 2, wherein the co-incubation is performed in a 1:0.5 to a 1:10 target antigen expressing cells to CAR expressing cells ratio.

4. The method of any of claims 1-3, wherein the cells analysis is performed by cell sorting analysis, preferably flow-cytometry analysis.

5. The method of any of claims 1-4, further comprising a step of incubating target antigen expressing cells with an antibody that recognizes the same or an overlapping epitope compared to the antibody from which the CAR is derived before co-incubating the labelled target antigen cells and the labelled CAR expressing cells.

6. The method of any of claims 1-5, wherein the antibody is a monoclonal antibody, preferably a monoclonal antibody that comprises CDRs of the monoclonal antibody from which the CAR is derived.

7. The method of any of claims 1-6, wherein the method further comprises testing control cells that do not express the targeted antigen.

8. The method of any of claims 1-7, wherein the method further comprises testing control CAR expressing cells that do not recognize the targeted antigen.

9. The method of any of claims 1-8, further comprising the selection of the functional CAR expressing cells and/or CAR construct.

10. The method of any of claims 1-9, wherein the labels are membrane markers, preferably lipophilic tracers or dyes, even more preferably selected from the group consisting of MIN126, PKH26-PCL, PKH67, MIN167, PKH67-PCL, PKH26, PKH26-PCL, Vybrant CM-Dil, Dil and DiO.

11. The method of any of claims 1-10, wherein the immune cells are selected from the group consisting of T lymphocytes, B lymphocytes, natural killer cells, natural killer T cells, monocytes and antigen presenting cells.

12. An in vitro method for selecting functional CAR expressing cells for adoptive cell therapy, which method comprises:
(i) transducing the population of immune cells with a nucleic acid sequence encoding a CAR, preferably wherein the population of immune cells has been isolated from a biological sample from a subject to be treated,
(ii) selecting a subpopulation of said isolated cells expressing the CAR and,
(iii) assessing their functionality by the method according to any of the preceding claims, preferably towards target antigen expressing cells that expresses the antigen for which the CAR has been generated
(v) optionally selecting the functional CAR expressing cells and/or the CAR nucleic acid construct from the CAR expressing cells that experienced the membrane acquisition form the antigen expressing cells.

13. The method of any of claims 1-12, wherein the antigen is HLA-G, preferably HLA-G1.
